(19) United States
(12) Patent Application Publication
(10) Pub. No.: US 2008/0045870 A1
(43) Pub. Date: Feb. 21, 2008

(54) SLING HOLDER

(76) Inventors: Marcia Grossman Nozik, West Hartford, CT (US); Randy Grossman Nozik, West Hartford, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/853,105

(22) Filed: Sep. 11, 2007

Related U.S. Application Data
(63) Continuation-in-part of application No. 10/759,713, filed on Jan. 16, 2004, now abandoned.
(60) Provisional application No. 60/440,990, filed on Jan. 17, 2003.

(51) Int. Cl. A61F 5/00; A61F 7/00

(52) U.S. Cl. 602/4; 607/112; 607/114

(57) ABSTRACT

A sling holder comprises a strap having first and second ends and a fastener for securing the strap to itself. The strap includes at least one pocket into which the object is placed. The strap ends are connected to one another, thereby locating and holding the object proximate to the skin in the desired position. The sling holder may be variable in length due to the selective attachment of a plurality of inter-engage able straps. Fasteners for coupling the straps are interposed between opposing ends of the assembled straps. If multiple straps are employed, any number of pockets may be attached to any number of the straps. An adjustable fastener, an elastic material, and/or a fixedly adjustable extension member may also be employed to adjust the length of the sling holder. The strap includes a contoured section of reduced width positioned between the first and second ends.

### CROSS REFERENCE TO RELATED APPLICATION

This application is a Continuation in Part of patent application Ser. No. 10/759,713 entitled “Sling Holder,” filed Jan. 16, 2004, which a utility application of Provisional Patent Application No. 60/440,990 filed on Jan. 17, 2003. The subject matter of these patent documents is incorporated by reference herein in its entirety.

### FIELD OF THE INVENTION

The invention generally relates to an apparatus for holding objects, and more specifically to a sling holder that is used for securing objects for extended periods of time against or proximate to surfaces of the skin.

### BACKGROUND OF THE INVENTION

There are a variety of reasons for desiring to temporarily hold an object for an extended period of time against or proximate to the skin. For example, when recovering from a wisdom tooth extraction, a cold pack placed against the skin of a person's cheek helps to reduce swelling and to relieve pain. In other cases, aromatics placed proximate to the skin near a nose may relieve congestion.

Generally, an object is conventionally temporarily placed for an extended period of time on or proximate to the skin by manually holding the object in a hand and moving the hand to the desired location. This permits proper positioning of the object. The hand also has the ability to control the pressure the object exerts on the skin and any underlying tissue, thereby minimizing any pain associated with placing and holding the object. Manually holding the object, however, often results in the hand (and possibly the arm and shoulder) becoming fatigued. Alternatively, the object may be held proximate to the skin using a sling system, which may alleviate at least some of the problems associated with manually holding the object against the skin. Such sling systems, however, often rely on pressing the object against the skin. Thus, considerable pressure can be exerted on an otherwise tender area increasing the level of discomfort.

Existing sling systems are furthermore typically manufactured in specific sizes and are therefore not adjustable so as to allow the same sling to be utilized by, for example, a small child and a large adult, or by the same person on different parts of that person's body. Thus, several different size sling systems must be considered for use within a family or even by one person. Also, most users tend to use an improperly-sized sling, which may also exert excess pressure on a tender area and exacerbate the level of discomfort.

Based on the foregoing, it is an object of the present invention to overcome the problems and drawbacks of the prior art.

### SUMMARY OF THE INVENTION

The invention resides in one aspect in a sling holder that when assembled can be secured around a body part to hold an object thereto. The sling holder comprises a strap having first and second ends and a fastener for securing the strap to itself. The strap includes at least one pocket into which the object is placed. The strap is placed around the body part and over the desired portion of the skin. The strap ends are connected to one another, thereby locating and holding the object proximate to the skin in the desired position. The strap includes a contoured section of reduced width disposed between the first end and second end. In one embodiment, at least one pocket is positioned between the contoured section of reduced width and at least one of the first end and the second end.

In another embodiment, the contoured section of reduced width includes a third end and a fourth end coupled to one another by a second fastener. The second fastener is positioned at the third end and the fourth end of the strap.

In another aspect of the invention, the sling holder is variable in length and width due to the selective attachment of the inter-engageable straps. In one embodiment, the sling holder includes a first strap and a second strap. A third strap may connect the first strap to the second strap, the third strap being removably attachable to the first strap and the second strap. Fasteners for coupling the straps are interposed between opposing ends of the assembled straps. Any number of pockets may be attached to any number of the straps. An adjustable fastener, an elastic material, and/or an adjustable extension member may also be employed to adjust the length of the sling holder.

The pocket either can be fixedly attached or removably attached by a fastener to any portion of a strap. If the pocket is movably attached, the fastener may allow for removal of the pocket from the strap with subsequent reattachment. The pocket can also be defined by any portion of the strap. The fastener may also allow for repositioning of the pocket on the strap.

Multiple fasteners may be provided to permit multiple pockets to be accommodated on the strap and to permit repositioning of the pockets relative to each other.

In situations where heat is to be transmitted from the object in the pocket to the skin of the body part, or from the body part to the object in the pocket, the pocket may include a wall that contacts the skin that is made from a thermally conductive material. Conversely, where transmission of heat is not desired, the pocket may include a wall made from insulating material.

The objects that may be inserted into the pocket can vary widely. For example, objects may include hot and/or cold packs, therapeutic magnets, or scented materials.

### BRIEF DESCRIPTION OF THE DRAWINGS

FIG. 1 is a front view of a sling holder in accordance with the present invention.

FIG. 2 is a back view of the sling holder of FIG. 1.

FIG. 3 is a top view of the sling holder of FIG. 1 in a coupled position.

FIG. 4 is a front view of an alternate embodiment of a sling holder.

FIG. 5 is a sectional view of the sling holder of FIG. 4.

FIG. 6 is a front view of another alternate embodiment of a sling holder.

FIG. 7 is an exploded front view of an adjustable length sling holder.

FIG. 8 is an exploded back view of the sling holder of FIG. 7.

FIG. 9 is an end view of the sling holder of FIG. 7 in a coupled position.

FIG. 10 is a front view of an alternate embodiment of a strap of the sling holder.

FIG. 11 is a sectional view of the strap of FIG. 10.

FIG. 12 is a front view of an alternate embodiment of a sling holder.

FIG. 13 is a back view of an alternate embodiment of a sling holder.

FIG. 14 is top schematic view if the sling holder of FIG. 12.

FIG. 15 is a detailed front back view of a portion of the strap of FIG. 1.

FIG. 16 is a detailed front back view of another embodiment of a portion of the strap of FIG. 1.

FIG. 17 is a detailed front back view of another embodiment of a portion of the strap of FIG. 1.

### DETAILED DESCRIPTION AND PREFERRED EMBODIMENTS

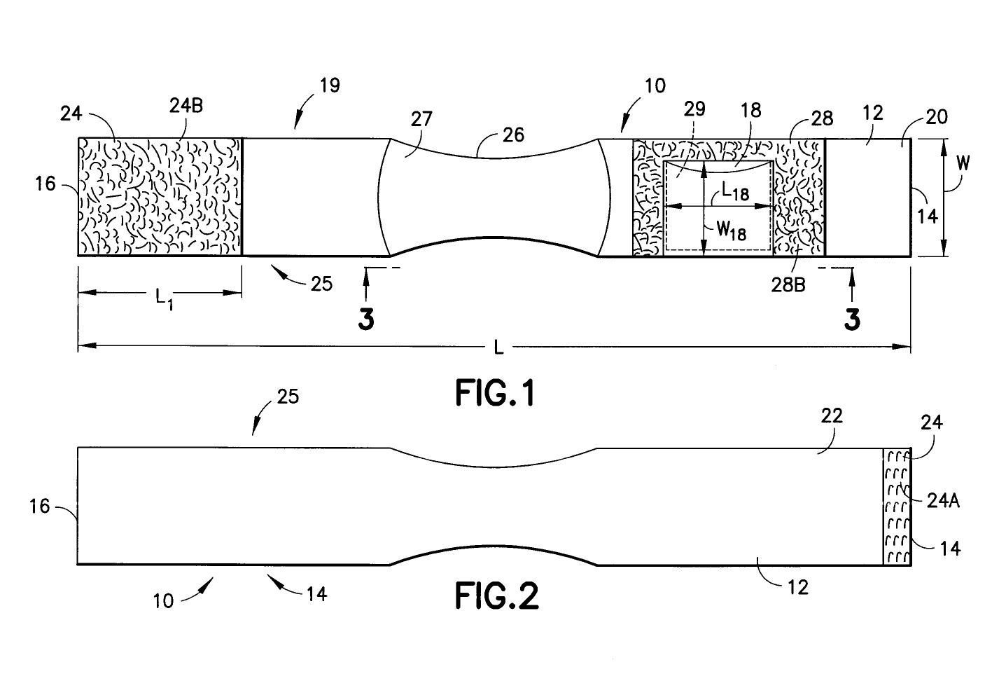

As shown in FIGS. 1-3, a sling holder, generally denoted by reference number 10, includes a strap 12 of length L and width W and having a first end 14 and an opposing second end 16. The strap 12 also includes one or more pockets 18, which may be fixedly or removably attached to the strap 12 or defined by the strap 12 itself. A single fastener 24 permits the first end 14 to be coupled to the second end 16.

The strap 12 has a perimeter defined by opposing sides 19, 25 of length L, first end 14 and second end 16, both of width W. The fastener 24 is positioned completely within the perimeter of the strap 12. In one embodiment, at least a portion of the fastener 24 abuts the first end 14 and/or the second end 16. In one embodiment the, fastener has a width equal to the width W of the strap 12. In one embodiment, at least a portion of the fastener 24 abuts at least a portion of at least one of the opposing sides 19, 25.

The strap 12 also has a first surface 20 and an opposingly positioned second surface 22. A contoured section 26 may be defined by the first surface 20 and the second surface 22 over a portion of the length L over which the width W may be narrowed. The contoured section is disposed between the first end 14 and the second end 16. The specific characteristics of the contoured section 26 (and whether the contoured section 26 is even provided) are based upon the proposed application for the sling holder. If provided, the contoured section 26 provides an improved fit of the strap 12 to a body portion. For example, where the sling holder 10 is positioned under a chin and over the top of a head, the strap 12 may be designed to eliminate or at least minimize local bunching under the chin.

In one embodiment, at least one of the pockets 18 is positioned between the contoured section 26 and the first end 14. In another embodiment, at least one of the pockets 18 is positioned between the contoured section 26 and the second end 16. In yet another embodiment, at least one of the pockets 18 is positioned between the contoured section 26 and the first end 14 and at least one additional pocket 18 is positioned between the contoured section 26 and the second end 16.

The fastener 24 is selectively fastenable to allow the length L of the sling holder 10 to be adjusted. In one exemplary embodiment, the fastener 24 comprises hook-and-loop fastening material, the hook portion being characterized as 24A and the loop portion being characterized as 24B. In such an embodiment, the loop portion 24B has a length L₁ that is longer than is necessary to effectively secure the hook portion 24A. As a result, the hook portion 24A can be affixed to the loop portion 24B at any one of a variety of locations over the length L₁ of the loop portion 24B. It should be understood that although a fastener of the hook-and-loop material type is shown, other practical fasteners, such as snaps, adhesive strips, and the like are considered within the scope of the present invention.

The strap 12 can be fabricated from any suitable synthetic or natural material. The material may be elastic, non-elastic, or a combination of elastic- and non-elastic materials. Exemplary materials for the strap 12 include, but are not limited to, rubber, elastomeric materials (e.g., neoprene), polyester, nylon, cotton, wool, rayon, bamboo, silk, and combinations of the foregoing materials. Straps made entirely from non-elastic materials are particularly desirable when it is desirable to minimize the pressure of an object 29 in the pocket 18 against the skin. The strap 12 may also have padding 27 that can be placed anywhere on the strap 12 based on the application for the sling holder 10.

The pocket 18, having a length L₁₈, a width W₁₈ and a depth suitable for holding the object 29, is preferably removably attachable to the first surface 20 of the strap 12 by a fastener 28. The fastener 28 may be of the hook-and-loop type with one portion (e.g., the hook portion) on the underside of the pocket 18 and another portion 28B (e.g., the loop portion) on the first surface 20. As a result, the pocket 18 can adopt any orientation on the surface 20 that is desired by the user. Furthermore, the loop portion 28B can be located anywhere on the first surface 20 so as to facilitate the placing of the pocket 18 anywhere on the strap 12.

The loop portion 28B preferably has a surface area that is greater than a surface area of the back wall of the pocket 18. As a result, the pocket 18 may be located at different locations along the length L or width W of the strap 12 within the loop portion 28B. While fasteners of the hook-and-loop type have been shown, other fasteners such as snaps, single-use adhesive strips, reusable adhesive strips, or the like can be used without departing from the scope of the present invention. Additionally, the straps can be fastened with stitching or glue. The dimensions of the pocket 18 depend on the intended use of the sling holder 10 and are based on the type of object 29 held by the sling holder 10.

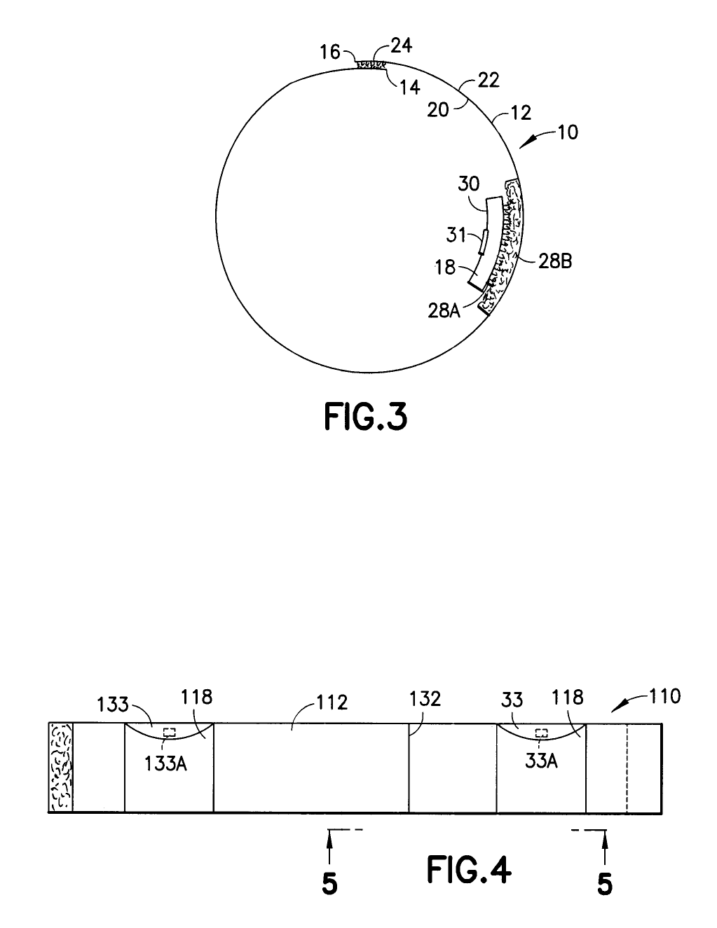

Referring specifically to FIG. 3, the hook portion and the loop portion of the fastener 24 are disposed on the first surface 20 and the opposingly positioned second surface 22 to allow the sling holder 10 to be coupled and secured to a body portion of a user. The hook portion of the fastener 28 for the pocket is shown at 28A and is positioned on the back wall of the pocket 18 such that when the fastener 24 is engaged to close the sling holder 10, a front wall 30 of the pocket 18 is facing inwardly of the sling holder 10. This permits the front wall 30 to be brought into contact with and to be held against the skin of the user.

When conduction of heat through the front wall 30 is desired, such as when the object in the pocket 18 is a hot or cold pack, the front wall 30 may be made from a thermally conducting material. The front wall 30, as well as other parts of the pocket 18, may have a temperature indicator 31, such as a temperature sensitive material that changes color as a function of temperature, to indicate the temperature of the hot or cold pack as well as to provide aesthetic appeal to the user. A temperature indicator may also be disposed on the strap 12. Conversely, when thermal conduction is not desired, the front wall 30 may be made of an insulating material to inhibit the conduction of heat therethrough.

Referring now to FIG. 4, another embodiment of the sling holder is indicated generally by the reference number 110. The sling holder 110 includes two pockets 118 attached to a strap 112. To adjust the length of the strap 112, an extension member 132 is provided on the strap 112. As can be seen in FIG. 5, the extension member 132 employs a temporary fastener 134, e.g., hook-and-loop fastening material mounted on interfacially engaging portions of the strap 112. Fasteners other than hook-and-loop material may be used, such as snaps, adhesive strips, buttons and button hole configurations, zippers (with or without pulls), combinations of the foregoing fasteners, and the like without departing from the scope of the invention. Furthermore, the pockets 118 may be fixedly attached to the strap 112. A flap 133 (optionally having a fastener 133A) may be provided to secure objects placed in the pocket 118.

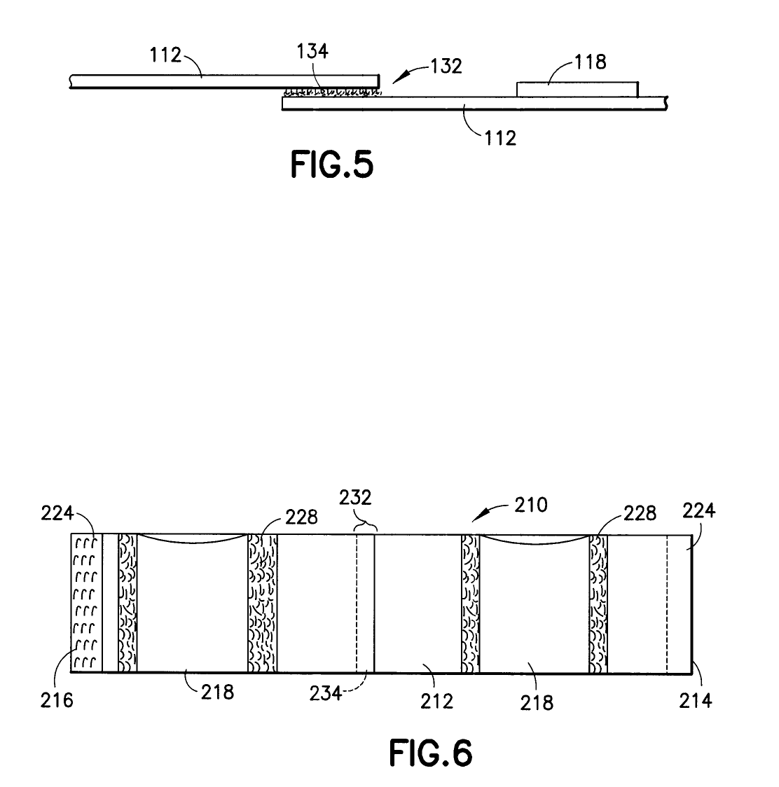

Referring now to FIG. 6, another embodiment of a sling holder in accordance with the present invention is indicated generally by the reference number 210. The sling holder 210 has two pockets 218 attached to a strap 212. The pockets 218 may be attached to the strap 212 using fasteners 228 of, for example, hook-and-loop fastening material so as to permit the relocation of the pockets 218 on the strap 212. A fastener 224 that couples a first end 214 and an opposing second end 216 of the strap 212 to each other are preferably also of the hook-and-loop fastening material type and are sized to permit length adjustment of the strap 212. Additionally, an extension member 232 also employing a fastener 234 of the hook-and-loop material type may be provided. In such an embodiment, a length of the strap 212 can be adjusted either using the fastener 224 or the extension member 232. Additionally, the spatial relationships of the pockets 218 one to the other can be adjusted either by adjusting the extension member 232 or by selective placement of the pockets 218 on the strap 212.

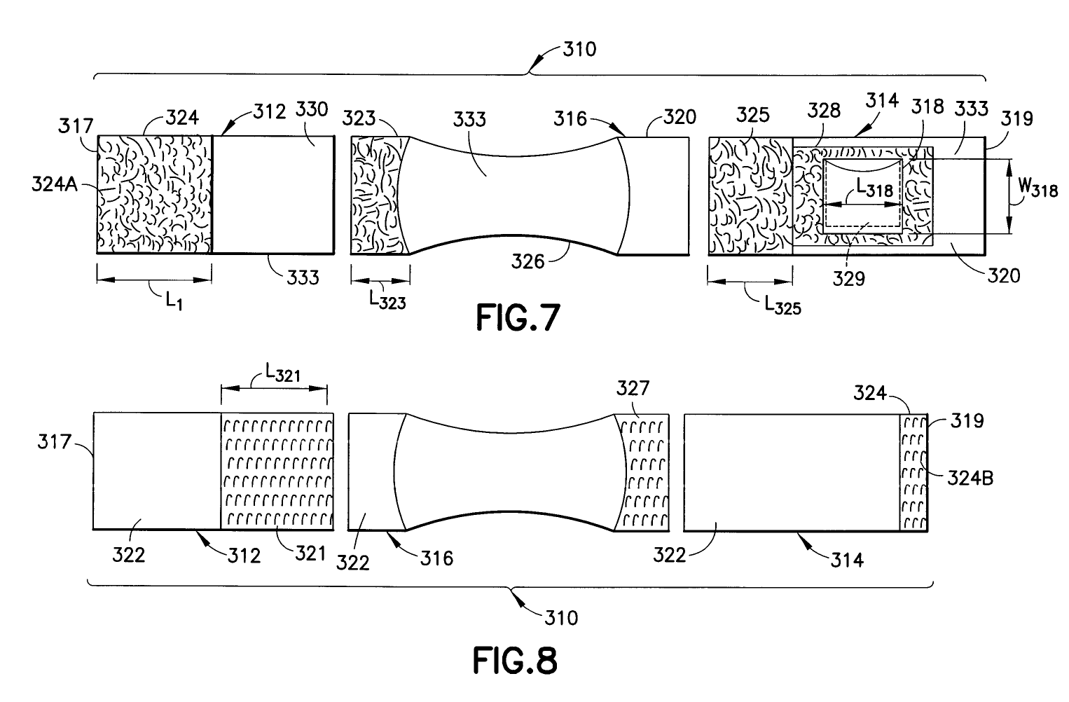

Referring now to FIGS. 7-9, a sling holder 310 may be configured to be adjustable by the incorporation of a plurality of engagable straps. In the embodiment shown in FIGS. 7 and 8, the adjustable sling holder includes three straps, namely, a first strap 312, a second strap 314, and a third strap 316. It should be understood, however, that any number of straps may be assembled to form the sling holder 310. The third strap 316 is interposed between and connects the first strap 312 to the second strap 314. A pocket 318 is disposed at at least one of the first strap 312, the second strap 314, and the third strap 316. As shown in FIG. 7, for example, the pocket 318 is attached to the second strap 314. When the first strap 312, the second strap 314, and the third strap 316 are assembled to form the sling holder 310, a first end 317 of the sling holder 310 is defined by the first strap 312 and an opposing second end 319 is defined by the second strap 314. Also defined by the assembled sling holder 310 are a first surface 320 and an opposing second surface 322. A fastener 324 permits the first end 317 to be coupled to the second opposing end 319.

The first strap 312, the second strap 314, and the third strap 316 are attachable at their respective ends using any suitable fastener. As with the embodiments disclosed above, a suitable fastener may be hook-and-loop fastening material attached to the first surface 320 and the opposing second surface 322 at the ends of each strap such that upon assembly of the straps to form the sling holder 310, the hook-and-loop fastening material cooperates to maintain the structural integrity of the sling holder 310. As is shown, the first strap 312 includes a hook portion 321 at the end thereof that cooperates with a loop portion 323 on the third strap 316. The hook portion 321 preferably has a length L₃₂₁ that enables the loop portion 323 to be selectively placed thereon to facilitate variably overlapping the first strap 312 and the third strap 316 to thereby provide for the adjustment of the length of the sling holder 310. The second strap 314 includes a loop portion 325 at an end thereof that cooperates with a hook portion 327 on the third strap 316. The loop portion 325 preferably has a length L_{0.325} that enables the hook portion 327 to be selectively placed thereon to facilitate variably overlapping the second strap 314 and the third strap 316 to thereby provide for further adjustment of the length of the sling holder 310. Alternatively, the first strap 312 can be connected to the second strap 314 without the third strap 316, thereby minimizing the length of the sling holder 310.

The third strap 316 may also have a contoured section 326 over a portion of its length that becomes narrow over the width of the sling holder 310. The shape of the contoured section 326 is based on the proposed application for the sling holder 310 to facilitate the positioning of the third strap 316 by providing a better fit of the sling holder 310 to a body portion.

To provide overall length adjustment to the sling holder 310, at least one portion of the fastener 324, e.g., a loop portion 324A of hook-and-loop fastening material, has a length that provides for the effective securing of a hook portion 324B. As a result, the hook portion 324B can be affixed to the loop portion 324A at any location over its length. While a fastener of the hook-and-loop type is shown, other practical fasteners such as snaps or adhesive strips may be substituted without departing from the scope of the present invention.

The third strap 16 may also have padding 333 which can be placed anywhere on the sling holder 310, such as on the first strap 312, the second strap 314, or both, based on the application for the sling holder 310.

The pocket 318, having a length L₃₁₈, a width W₃₁₈, and a depth suitable for holding an object 329, is preferably removably secured to the first surface 320 of the sling holder 310 by a fastener 328. Although the pocket 318 is shown as being mounted on the second strap 314, it should be understood that the pocket 318 may alternatively be mounted on the first strap 312 or on the third strap 316. Furthermore, any number of pockets 318 may be mounted on any one or any number of straps.

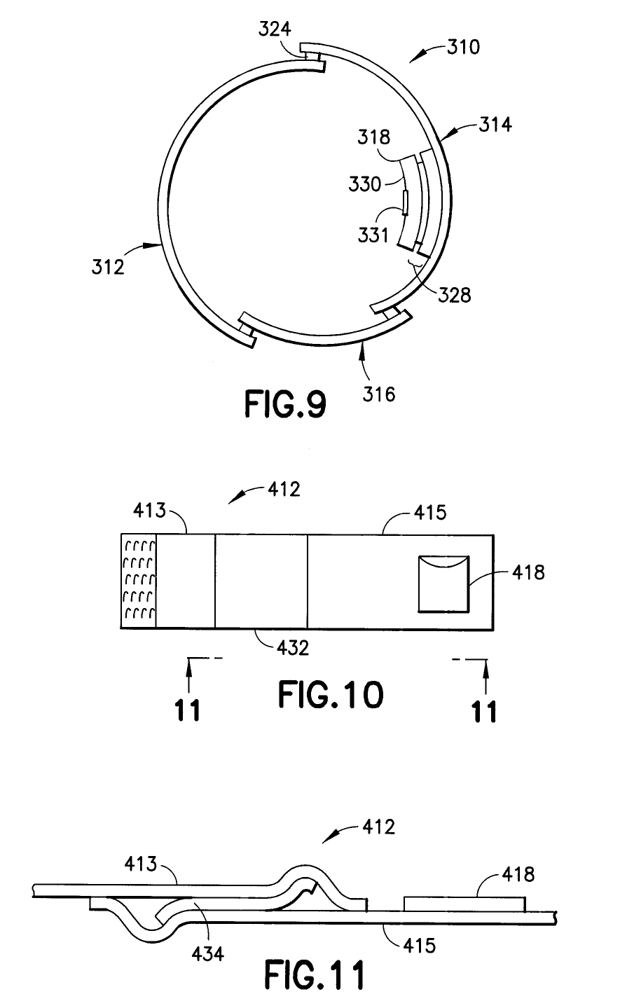

Referring to FIG. 9, the hook portion and the loop portion are disposed on opposite sides of the first surface and the second opposing surface in order to be coupled to one another to secure the sling holder 310 to a body portion of a user. The fastener 328 for the pocket 318 is positioned on a surface of the strap 314 such that when the fastener 324 is engaged to close the sling holder 310, a surface 330 of the pocket 318 is within the interior of the closed sling holder 310. This permits the wall 330 to be brought into contact with and to be held against the skin of the user. As above, the wall 330, as well as other parts of the pocket 318, may have a temperature indicator 331, such as a temperature sensitive material that changes color as a function of temperature, to indicate the temperature of the hot or cold pack as well as to provide aesthetic appeal to the user. A temperature indicator may also be disposed on at least one of the first strap 12, the second strap 14, and the third strap 16.

Referring now to FIGS. 10 and 11, another embodiment of a strap for a sling holder is indicated generally by the reference number 412. The strap 412 is similar to the first strap 312 as described with reference to FIGS. 7-9 (e.g., it may include a pocket 418) and may be used with at least one other strap to provide a variable-length sling holder. The strap 412 includes a first portion 413 and a second portion 415 connected by at least one extension member 432. The extension member 432 provides for length adjustment of an individual strap. As shown in FIG. 11, the extension member 432 employs a temporary fastener 434, such as a hook and loop fastening material mounted on the interfacially engaging first portion 413 and second portion 415 of the strip 412. To expand the strap 412 using the extension member 432, the interfacially engaging portions of the strap 412 are disengaged and subsequently re-engaged at a different location. As fasteners 434 other than the hook-and-loop type may be used, such as adhesive strips, buttons with button holes, snaps, and zippers (with or without pulls), the invention should not be considered so limited.

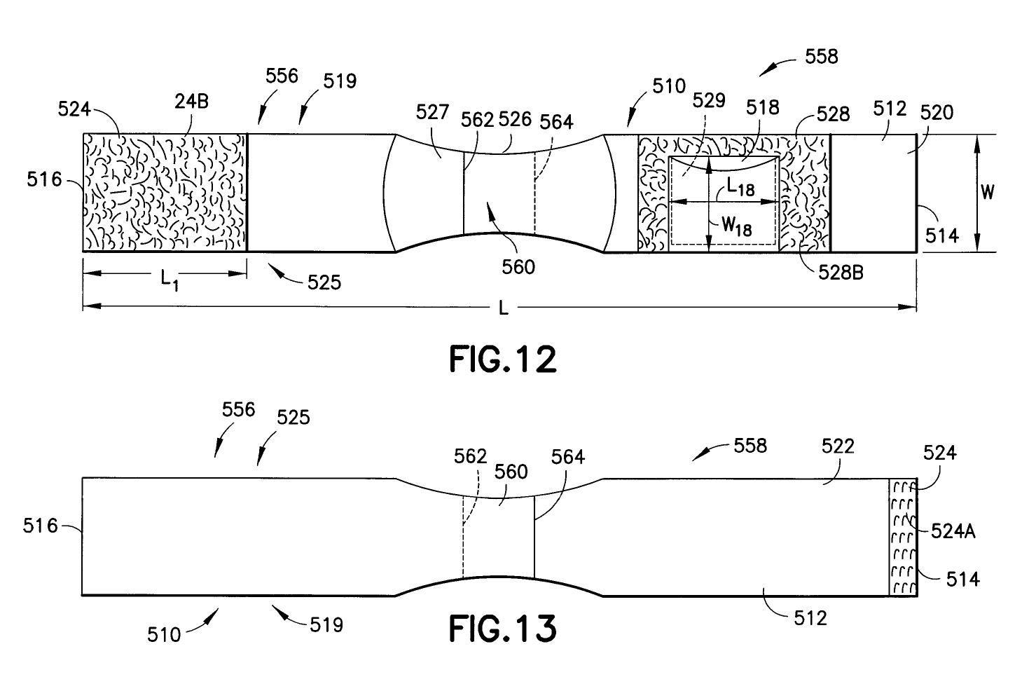

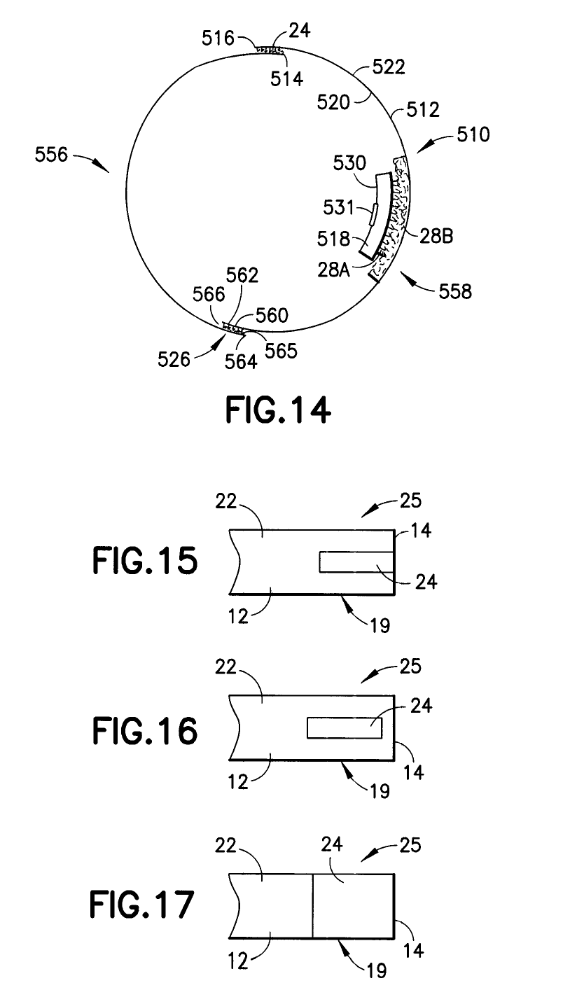

Referring to FIGS. 12-14, another embodiment of a strap 512 for a sling holder 510 is indicated generally by the reference number 512. The strap 512 is similar to the strap 12 illustrated in FIGS. 1-3 thus similar elements are assigned similar reference numerals preceded by the numeral 5. The sling holder 510 includes a strap 512 of length L and width W and having a first end 514 and an opposing second end 516. The strap 512 also includes one or more pockets 518, which may be fixedly or removably attached to the strap 512 or defined by the strap 512 itself. A single fastener 524 permits the first end 514 to be coupled to the second end 516.

The strap 512 has a perimeter defined by opposing sides 519, 525 of length L, first end 514 and second end 516, both of width W. The fastener 524 is positioned completely within the perimeter of the strap 512. In one embodiment, at least a portion of the fastener 524 abuts the first end 514 and/or the second end 516. In one embodiment the, fastener has a width equal to the width W of the strap 512. In embodiment, at least a portion of the fastener 524 abuts at least a portion of at least one of the opposing sides 519, 525.

The strap 512 also has a first surface 520 and an opposingly positioned second surface 522. A contoured section 526 may be defined by the first surface 520 and the second surface 522 over a portion of the length L over which the width W may be narrowed. The contoured section is disposed between the first end 14 and the second end 516. The specific characteristics of the contoured section 526 (and whether the contoured section 26 is even provided) are based upon the proposed application for the sling holder. If provided, the contoured section 526 provides an improved fit of the strap 512 to a body portion. For example, where the sling holder 510 is positioned under a chin and over the top of a head, the strap 512 may be designed to eliminate or at least minimize local bunching under the chin.

In one embodiment, at least one of the pockets 518 is positioned between the contoured section 526 and the first end 514. In another embodiment, at least one of the pockets 518 is positioned between the contoured section 526 and the second end 516. In yet another embodiment, at least one of the pockets 518 is positioned between the contoured section 526 and the first end 514 and at least one additional pocket 518 is positioned between the contoured section 526 and the second end 516.

The strap 512 is comprised of a first strap portion 556 and a second strap portion 558, removably coupled to one another by a second fastener 560 to allow the sling holder 510 to be removed, installed and/or adjusted in length, for example, about the chin of a user. The contoured section 526 includes a third end 562 disposed on the second strap portion and fourth end 564 disposed on the first strap portion 556 each defining a portion of the second fastener 560.

The fastener 560 comprises hook-and-loop fastening material, the hook portion being characterized as 565 and the loop portion being characterized as 566. In such an embodiment, the loop portion 566 has a length that is longer than is necessary to effectively secure the hook portion 565. As a result, the hook portion 565 can be affixed to the loop portion 566 at any one of a variety of locations over the length of the loop portion 566. It should be understood that although a fastener of the hook-and-loop material type is shown, other practical fasteners, such as snaps, adhesive strips, and the like are considered within the scope of the present invention.

Referring to FIG. 15, at least a portion of the fastener 24 abuts the first end 14. The fastener 24 abutting the second end 16, is also within the scope of the present invention. The fastener 24 illustrated in FIG. 16, is positioned completely within the perimeter of the strap 12. In one embodiment, fastener 24 illustrated in FIG. 17 has a width equal to the width W of the strap 12. While the above embodiments are described, the present invention is not limited in this regard as at least a portion of the fastener 24 abutting at least a portion of at least one of the opposing sides 19, 25, is also within the scope of the present invention.

While the present invention has been described in considerable detail with reference to certain preferred embodiments thereof, other embodiments are possible. Therefore, the scope of the invention should not be limited to the description of the preferred versions contained herein.

1. A sling holder for holding an object for an extended period of time against the skin, said sling holder comprising:
a strap having a first end and an opposing second end;
said strap having a contoured section of reduced width disposed between the first end and the second end;
a single fastener for coupling said first end to said second end, said fastener being positioned at said first end and said opposing second end of said strap and positioned completely within the perimeter of said strap; and
at least one pocket attachable to said strap.

2. The sling holder of claim 1, wherein said at least one pocket is positioned between the contoured section of reduced width and at least one of the first end and the second end.

3. The sling holder of claim 1, wherein said contoured section of reduced width comprises a third end and a fourth end coupled to one another by a second fastener, said second fastener being positioned at said third end and said fourth end of said strap.

4. The sling holder of claim 1, wherein said fastener is selectively fastenable to allow a length of said sling holder to be adjusted.

5. The sling holder of claim 1, wherein said fastener is hook-and-loop fastening material.

6. The sling holder of claim 1, further comprising padding disposed on said strap.

7. The sling holder of claim 1, wherein said pocket is removably attachable to said strap.

8. The sling holder of claim 1, wherein said pocket has an inwardly facing surface to be held against the skin of a user upon assembly of said sling holder on a body part.

9. The sling holder of claim 8, wherein said inwardly facing surface is fabricated from a thermally conducting material.

10. The sling holder of claim 8, wherein said inwardly facing surface is fabricated from a thermally insulating material.

11. The sling holder of claim 1, further comprising a temperature indicator on at least one of said strap and said pocket.

12. The sling holder of claim 1, further comprising a plurality of pockets to be attachable to said strap.

13. The sling holder of claim 1, wherein said strap includes an extension member interposed between ends of said strap for varying the length of said strap.

14. The sling holder of claim 13, wherein said extension member comprises a temporary fastener mounted on interfacially engaging portions of said strap.

15. A sling holder for holding an object against the skin, said sling holder comprising:
a first strap having a first end and an opposing second end;
said first strap having a contoured section of reduced width disposed between the first end and the second end;
a second strap for adjusting a length of said sling holder, said second strap being attachable at a first end thereof to said first strap;
a single fastener for coupling a second end of said second strap to said first strap, said fastener being positioned at said first strap and said second end of said second strap and positioned completely within the perimeter of said first and second straps; and
at least one pocket attachable to at least one of said first strap and said second strap.

16. The sling holder of claim 15, further comprising a third strap interposable between said first strap and said second strap for further adjusting said length of said sling holder.

17. The sling holder of claim 15 wherein said third strap is removably coupled to each of said first strap and said second strap using a hook-and-loop material.

18. The sling holder of claim 15, wherein said at least one pocket is positioned between the contoured section of reduced width and at least one of the first end and the second end.

19. The sling holder of claim 15, wherein said first strap includes:
a first strap portion having a third end and a second strap portion having a fourth end;
said third end and said fourth end being positioned in said contoured section of reduced width;
a second fastener coupling said first strap portion and said second strap portion to one another; and
said second fastener being positioned at said third end and said second end.

20. A sling holder for holding an object for an extended period of time against the skin, said sling holder consisting of:
a strap having a perimeter defined by opposing longitudinal sides, a first end and an opposing second end;
a single fastener for coupling said first end to said second end, said fastener being positioned at said first end and said opposing second end of said strap and positioned completely within the perimeter of said strap; and
at least one pocket attachable to said strap.
